(19) United States
(12) Patent Application Publication
(10) Pub. No.: US 2025/0378959 A1
(43) Pub. Date: Dec. 11, 2025

(54) EXPLAINABLE ARTIFICIAL INTELLIGENCE METHOD APPLIED TO CLINICAL MEDICINE AND SYSTEM THEREOF AND NON-TRANSITORY COMPUTER READABLE RECORDING MEDIUM

(71) Applicant: China Medical University, Taichung City (TW)

(72) Inventors: Chin-Chi Kuo, Taichung City (TW); Yu-Ting Lin, Taichung City (TW); Yi-Chun Chen, Taichung City (TW); Min-Yen Wu, Taichung City (TW)

(73) Assignee: China Medical University, Taichung City (TW)

(21) Appl. No.: 18/906,484

(22) Filed: Oct. 4, 2024

(30) Foreign Application Priority Data
Jun. 7, 2024 (TW) 113121244

(51) Int. Cl. G16H 50/30

(52) U.S. Cl. CPC G16H 50/30

(57) ABSTRACT

An explainable artificial intelligence method applied to clinical medicine includes reading a parameter dataset, a machine learning model, a model explainable program and a plurality of clinical index range values, the parameter dataset includes a plurality of parameters; inputting the parameter dataset into the machine learning model to generate a predicting result; executing the model explainable program to the machine learning model, to calculate a plurality of important values and a plurality of risk indexes; determining whether one of the parameters being out of one of the clinical index range values; comparing the parameters and the risk indexes to generate a risk information; comparing the parameters and the clinical index range values, and dividing the parameters into a plurality of trusting levels; and integrating the parameters, the important values, the risk information and the trusting levels into a visualization information.

### RELATED APPLICATIONS

This application claims priority to Taiwan Application Serial Number 113121244, filed Jun. 7, 2024, which is herein incorporated by reference.

### BACKGROUND

### Technical Field

The present disclosure relates to an explainable artificial intelligence method and a system thereof and a non-transitory computer readable recording medium. More particularly, the present disclosure relates to an explainable artificial intelligence method applied to clinical medicine and a system thereof and a non-transitory computer readable recording medium.

### Description of Related Art

Nowadays, medical science often combines with artificial intelligence method or machine learning method, in order to assist the medical personnel to make treatment decisions, and enhance disease management in clinical medicine. However, the artificial intelligence method or the machine learning method has a “black box” characteristic, the medical personnel can only obtain the predicting result or the advising decision calculated by the artificial intelligence method or the machine learning method, but cannot interpret or speculate the probably reason of the predicting result and verify an accuracy of the predicting result.

Thus, developing an explainable artificial intelligence method applied to clinical medicine and a system thereof and a non-transitory computer readable recording medium which can interpret and explain the predicting result is highly valuable.

### SUMMARY

According to one aspect of the present disclosure, an explainable artificial intelligence method applied to clinical medicine includes driving a processor to read a parameter dataset, a machine learning model, a model explainable program and a plurality of clinical index range values from a database, the parameter dataset includes a plurality of parameters, and the clinical index range values are corresponding to the parameters, respectively; driving the processor to input the parameter dataset into the machine learning model to generate a predicting result; driving the processor to execute the model explainable program to the machine learning model, to calculate a plurality of important values correspond to the parameters and a plurality of risk indexes; driving the processor to determine whether one of the parameters being out of one of the clinical index range values corresponding to the one of the parameters; driving the processor to compare the one of the parameters and one of the risk indexes to generate a risk information; driving the processor to compare the parameters and the clinical index range values, and divide the parameters into a plurality of trusting levels according to a consistency between the parameters and the clinical index range values, the one of the parameters corresponds to one of the trusting levels; and driving the processor to integrate the parameters, the important values corresponding to the parameters, the risk information and the trusting levels corresponding to the parameters into a visualization information.

According to another aspect of the present disclosure, an explainable artificial intelligence system applied to clinical medicine includes a database and a processor. The database is configured to access a parameter dataset, a machine learning model, a model explainable program and a plurality of clinical index range values. The parameter dataset includes a plurality of parameters, and the clinical index range values are corresponding to the parameters, respectively. The processor is signally connected to the database, reads the parameter dataset, the machine learning model, the model explainable program and the clinical index range values. The processor is configured to perform an explainable artificial intelligence method applied to clinical medicine. The explainable artificial intelligence method applied to clinical medicine includes inputting the parameter dataset into the machine learning model to generate a predicting result; executing the model explainable program to the machine learning model to calculate a plurality of important values correspond to the parameters and a plurality of risk indexes; determining whether one of the parameters being out of one of the clinical index range values corresponding to the one of the parameters; comparing a difference value between the one of the parameters and one of the risk indexes to generate a risk information; comparing the parameters and the clinical index range values, and dividing the parameters into a plurality of trusting levels according to a consistency between the parameters and the clinical index range values, the one of the parameters corresponds to one of the trusting levels; and integrating the parameters, the important values corresponding to the parameters, the risk information and the trusting levels corresponding to the parameters into a visualization information.

According to one aspect of the present disclosure, a non-transitory computer readable recording medium storing a program for a processor capable of generating a visualization information, to execute an explainable artificial intelligence method applied to clinical medicine. The explainable artificial intelligence method applied to clinical medicine includes driving the processor to read a parameter dataset, a machine learning model, a model explainable program and a plurality of clinical index range values from a database, the parameter dataset includes a plurality of parameters, and the clinical index range values are corresponding to the parameters, respectively; driving the processor to input the parameter dataset into the machine learning model to generate a predicting result; driving the processor to execute the model explainable program to the machine learning model, to calculate a plurality of important values correspond to the parameters and a plurality of risk indexes; driving the processor to determine whether one of the parameters being out of one of the clinical index range values corresponding to the one of the parameters; driving the processor to compare the one of the parameters and one of the risk indexes to generate a risk information; driving the processor to compare the parameters and the clinical index range values, and divide the parameters into a plurality of trusting levels according to a consistency between the parameters and the clinical index range values, the one of the parameters corresponds to one of the trusting levels; and driving the processor to integrate the parameters, the important values corresponding to the parameters, the risk information and the trusting levels corresponding to the parameters into the visualization information.

### BRIEF DESCRIPTION OF THE DRAWINGS

The present disclosure can be more fully understood by reading the following detailed description of the embodiment, with reference made to the accompanying drawings as follows:

FIG. 1 shows a block diagram of an explainable artificial intelligence system applied to clinical medicine according to a first embodiment of the present disclosure.

FIG. 2 shows a flow chart of an explainable artificial intelligence method applied to clinical medicine according to a second embodiment of the present disclosure.

FIG. 3 shows a schematic view of a visualization information generated by the explainable artificial intelligence method applied to clinical medicine of FIG. 2.

FIG. 4 shows a schematic view of another visualization information generated by the explainable artificial intelligence method applied to clinical medicine of FIG. 2.

FIG. 5A shows a flow chart of a part of an explainable artificial intelligence method applied to clinical medicine according to a third embodiment of the present disclosure.

FIG. 5B shows a flow chart of another part of the explainable artificial intelligence method applied to clinical medicine according to the third embodiment of the present disclosure.

FIG. 6 shows a schematic view of a visualization information generated by the explainable artificial intelligence method applied to clinical medicine of FIG. 5A and FIG. 5B.

FIG. 7 shows a schematic view of another visualization information generated by the explainable artificial intelligence method applied to clinical medicine of FIG. 5A and FIG. 5B.

FIG. 8 shows a schematic view of further another visualization information generated by the explainable artificial intelligence method applied to clinical medicine of FIG. 5A and FIG. 5B.

### DETAILED DESCRIPTION

The embodiment will be described with the drawings. For clarity, some practical details will be described below. However, it should be noted that the present disclosure should not be limited by the practical details, that is, in some embodiments, these practical details may be unnecessary. In addition, for simplifying the drawings, some conventional structures and elements will be simply illustrated, and repeated elements may be represented by the same labels.

It will be understood that when an element (or device) is referred to as be “connected to” another element, it can be directly connected to other element, or it can be indirectly connected to the other element, that is, intervening elements may be present. In contrast, when an element is referred to as be “directly connected to” another element, there are no intervening elements present. In addition, the terms first, second, third, etc. are used herein to describe various elements or components, these elements or components should not be limited by these terms. Consequently, a first element or component discussed below could be termed a second element or component.

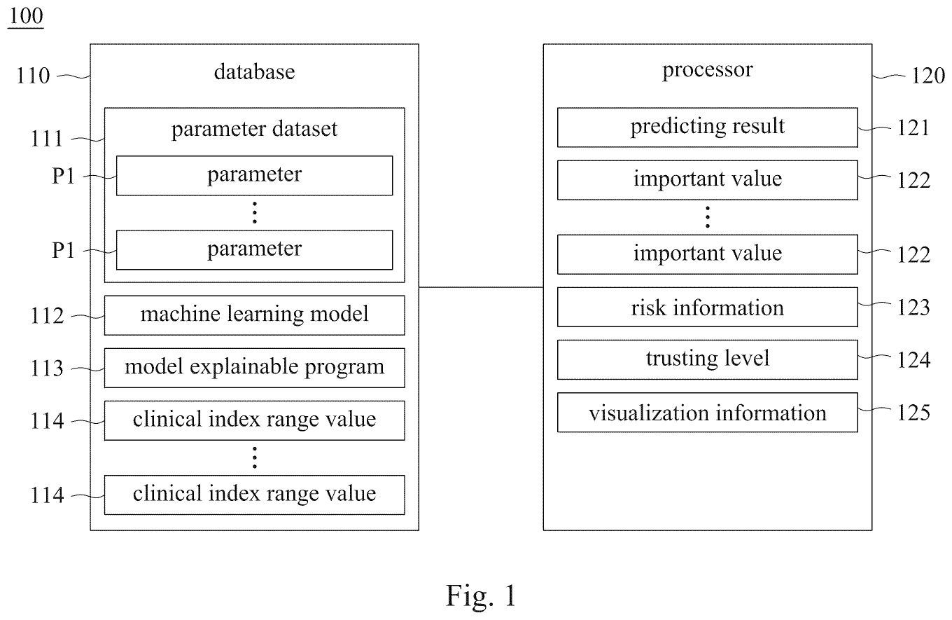

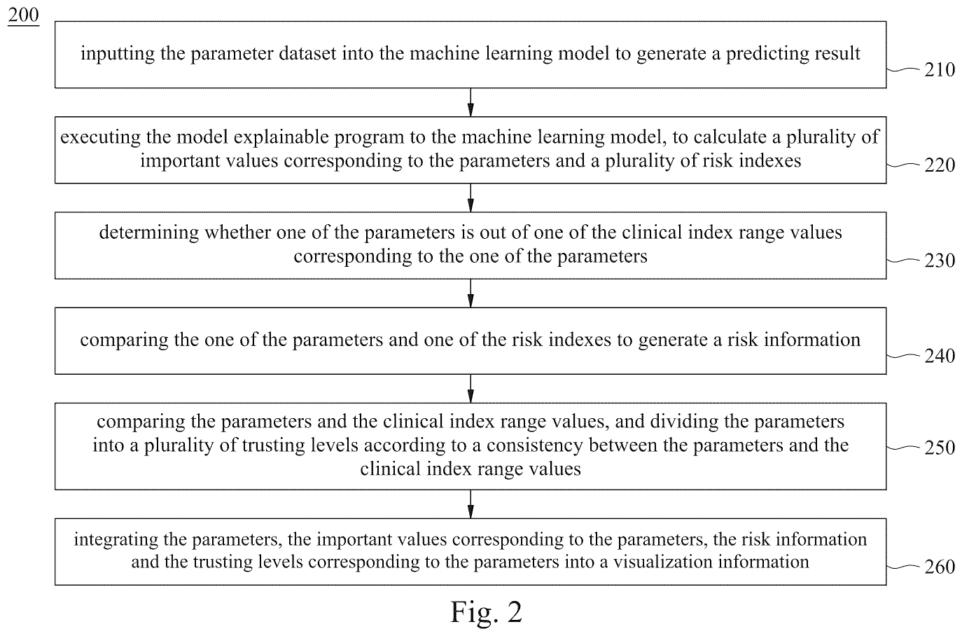

Please refer to FIG. 1. FIG. 1 shows a block diagram of an explainable artificial intelligence system 100 applied to clinical medicine according to a first embodiment of the present disclosure. The explainable artificial intelligence system 100 applied to clinical medicine includes a database 110 and a processor 120. The database 110 is configured to access a parameter dataset 111, a machine learning model 112, a model explainable program 113 and a plurality of clinical index range values 114. The parameter dataset 111 includes a plurality of parameters P1, and the clinical index range values 114 are corresponding to the parameters P1, respectively. The processor 120 is signally connected to the database 110, reads the parameter dataset 111, the machine learning model 112, the model explainable program 113 and the clinical index range values 114. The processor 120 is configured to perform an explainable artificial intelligence method 200 applied to clinical medicine (shown in FIG. 2).

In detail, the database 110 can include a Random Access Memory (RAM) capable to store information and instruction for the processor 120 to process or other dynamic storing device, the processor 120 can include any type of processor, microprocessor, the parameter dataset 111 can include medical parameters for training the machine learning model 112, the machine learning model 112 can include a model trained by any of a Transformed Fuzzy Neural Network (TFNN), a Deep Neural Network (DNN), an Integrated Genetic Algorithm and Support Vector Machine (IGS), an extreme Gradient Boosting (XGBoost), a Graph-based Class-Imbalanced Learning (Graph-CL), a Joint Imbalanced Classification and Feature Selection (JICFS), a Time Trajectory Learning (TTL), a Bi-directional Long Short-Term Memory (Bi-LSTM) and an Ensemble Model, but the present disclosure is not limited thereto. The model explainable program 113 can be one of a SHapley Additive explanation (SHAP), a Local Interpretable Model-Agnostic Explanations (LIME) and an Individual Conditional Expectation (ICE), but the present disclosure is not limited thereto.

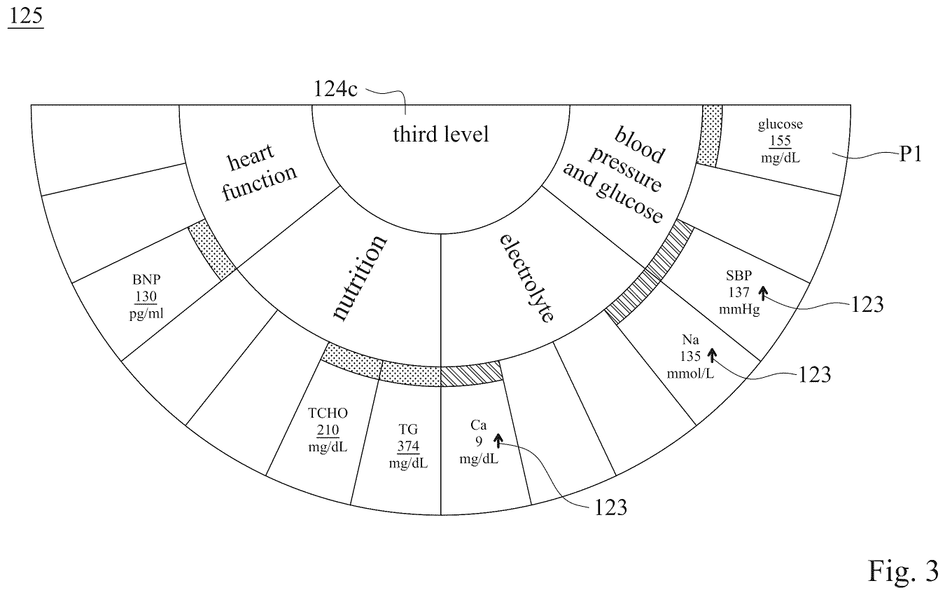

Please refer to FIG. 1 to FIG. 3. FIG. 2 shows a flow chart of an explainable artificial intelligence method 200 applied to clinical medicine according to a second embodiment of the present disclosure. FIG. 3 shows a schematic view of a visualization information 125 generated by the explainable artificial intelligence method 200 applied to clinical medicine of FIG. 2. The explainable artificial intelligence method 200 applied to clinical medicine includes steps 210, 220, 230, 240, 250, 260. The step 210 includes inputting the parameter dataset 111 into the machine learning model 112 to generate a predicting result 121. The step 220 includes executing the model explainable program 113 to the machine learning model 112, to calculate a plurality of important values 122 corresponding to the parameters P1 and a plurality of risk indexes. The step 230 includes determining whether one of the parameters P1 is out of one of the clinical index range values 114 corresponding to the one of the parameters P1 according to the clinical index range values 114. The step 240 includes comparing the one of the parameters P1 and one of the risk indexes to generate a risk information 123. The step 250 includes comparing the parameters P1 and the clinical index range values 114, and dividing the parameters P1 into a plurality of trusting levels 124 according to a consistency between the parameters P1 and the clinical index range values 114. The one of the parameters P1 corresponds to one of the trusting levels 124. The step 260 includes integrating the parameters P1, the important values 122 corresponding to the parameters P1, the risk information 123 and the trusting levels 124 corresponding to the parameters P1 into a visualization information 125. Thus, the explainable artificial intelligence system 100 applied to clinical medicine of the present disclosure can verify the predicting result 121 generated by the machine learning model 112, thereby, avoiding a situation of wrong medical decision and treatment due to mistaken predicting result 121.

Moreover, the step 210 is configured to input the parameter dataset 111, which is related to a disease to-be-predicted or a disease to-be-determined, to the machine learning model 112 transformed by an artificial intelligence method to generate a predicting result 121 related to the aforementioned disease. For example, the machine learning model 112 can be a predicting model for predicting a risk of Cardiovascular disease, the parameter dataset 111 can be the patient basic information, the vital signs, the physiological data of a clinic measurement, a medication list and a historical diagnostic information related to the risk of Cardiovascular disease prediction, the predicting result 121 can be a probability of a patient suffering from Cardiovascular disease, but the present disclosure is not limited thereto.

In the step 220, the relevance and the impact magnitude between the values of all the parameters P1 in the parameter dataset 111 and the predicting result 121 are calculated by the model explainable program 113, and the aforementioned relevance and the impact magnitude are transformed into an important value 122, which is corresponding to one of the parameters P1. A part of the parameters P1 in the parameter dataset 111 are shown in FIG. 3, the part of the parameters P1 include glucose, Systolic Blood Pressure (SBP), Natrium (Na), Calcium (Ca), Triglycerides (TG), Total Cholesterol (TCHO) and Brian Natriuretic Peptide (BNP).

In the step 230, the clinical index range value 114 can include a healthy value range of the parameter P1 in the parameter dataset 111, and the healthy value range can be determined by clinical trial or clinical practice experience. For example, the value of the SBP in FIG. 3 is 137 mmHg. In the database 110, a healthy range of the clinical index range value 114 corresponds to the SBP is under 140 mmHg. In FIG. 3, a value of the parameter P1 without underscore represents the value of the parameter P1 is in the healthy range, and the value of the parameter P1 with underscore represents the value of the parameter P1 is out of the healthy range. In other embodiments of the present disclosure, the value of the parameter can be marked with different colors to represent whether the value of the parameter is out of the healthy range, but the present disclosure is not limited thereto.

In the step 240, a risk index is generated according by the model explainable program 113, and a risk information 123 corresponding to the parameter P1 is generated according to a magnitude relationship between the value of the parameter P1 and the risk index. The predicting result 121 of the machine learning model 112 is analyzed by the model explainable program 113 to generate the risk index. The risk index is a critical value of each of the parameters P1, which can change the predicting result 121. In FIG. 3, the risk information 123 is represented by an up-pointing arrow or a down pointing arrow. The up-pointing arrow represents the value of the parameter P1 should be increased to decrease the risk of disease, and the down-pointing arrow represents the value of the parameter should be decreased to decrease the risk of disease. For example, when a value of “Ca” calculated by the model explainable program 113 is greater than 15 mg/dL, a value of the predicting result 121 (i.e., the risk of disease) can be decreased, that is, the risk index of parameter “Ca” is 15 mg/dL. In FIG. 3, the value (i.e., 9 mg/dL) of parameter “Ca” is less than 15 mg/dl, that is, the value should be increased to decrease the risk of disease, and the risk information 123 is represented by an up-pointing arrow.

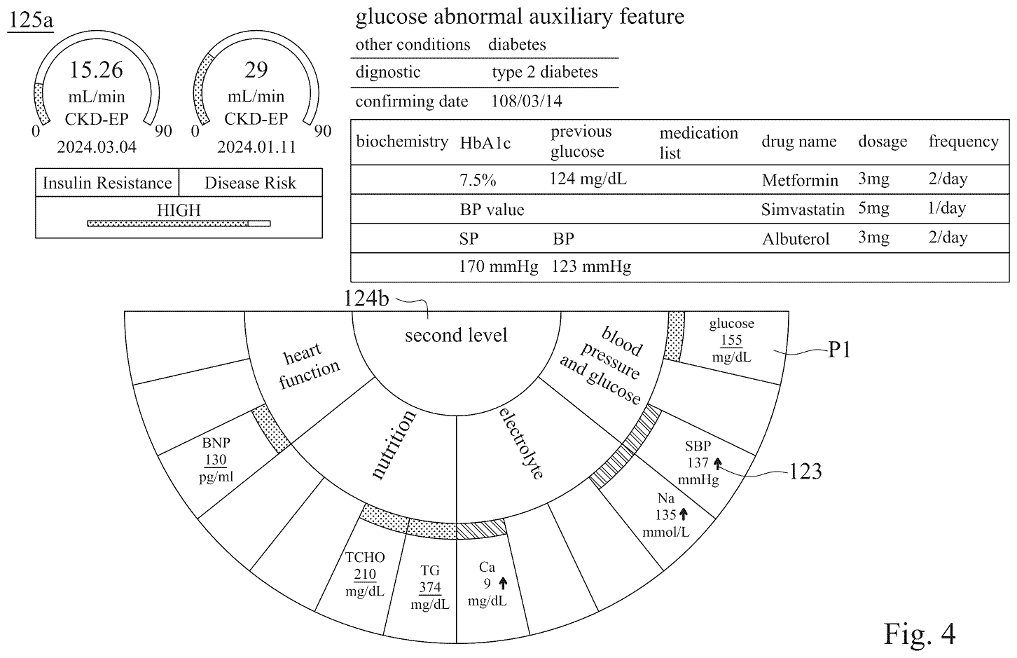

Please refer to FIG. 1 to FIG. 4. FIG. 4 shows a schematic view of another visualization information 125a generated by the explainable artificial intelligence method 200 applied to clinical medicine of FIG. 2. In the step 250, the trusting levels 124 can include a first level (its reference numeral is omitted in the second embodiment), a second level 124b and a third level 124c. The first level represents the result of the predicting result 121 being high risk or low risk and the result of the value of the parameter P1 exceeding the clinical index range value 114 or not is consistent, and the first level can be listed in Table 1.

TABLE 1

| | | |
|---|---|---|
| The consistency | The value of the | The value of the |
| between the predicting | parameter P1 exceeds | parameter P1 is in the |
| result 121 and the | the clinical index | clinical index range |
| clinical index range | range value 114 | value 114 |
| value 114 | | |
| The predicting result 121 | consistent | inconsistent |
| is high risk | | |
| The predicting result 121 | inconsistent | consistent |
| is low risk | | |

Please refer to FIG. 4, the second level 124b represents the result of the predicting result 121 generated by the machine learning model 112 being high risk or low risk and the result of the value of the parameter P1 exceeding the clinical index range value 114. However, after incorporating other auxiliary judgment features for evaluation, the clinical determination of disease risk may be inconsistent with the prediction result 121. In the clinical judgement of determining the risk of disease, besides determining whether a value of a single parameter P1 exceeding the clinical index range value 114 or not, other auxiliary features are also considered to determine the risk of disease. For instance, the value of glucose in FIG. 4 is 155 mg/dL, and the clinical index range value 114 of glucose is under 126 mg/dL. However, in the clinical judgement, the risk of disease cannot be only determined by the value of glucose. Other values of abnormal auxiliary features (such as diabetes, Glycated Hemoglobin (Hba1c) and the medication list of diabetes) should also be considered to determine the risk of disease. Thus, the parameter “glucose” is listed as a parameter P1 in the second level 124b. Moreover, if a patient corresponding to the parameter dataset 111 is not a diabetes patient, and does not take medicine of diabetes, the risk of diabetes can be judged without auxiliary features. The parameter “glucose” can be listed in the first level. If a patient has diabetes, and has taken medicine of diabetes, the value of other auxiliary feature (such as a value of Hba1c) exceeding the clinical index range value 114 or not should also be considered, and the parameter “glucose” is determined as a parameter P1 in the second level 124b (shown in FIG. 4) or a parameter P1 in the third level 124c according to the predicting result 121 and the risk of disease corresponding to the glucose.

Further, the third level 124c represents the result of the predicting result 121 being high risk or low risk and the value of the parameter P1 exceeding the clinical index range value 114 or not is inconsistent.

In the step 260, the parameters P1, which are corresponding to different trusting levels 124, the important values 122, which are corresponding to the parameters P1, are integrated to a visualization information 125a. Thus, the explainable artificial intelligence method 200 applied to clinical medicine of the present disclosure can enhance the physician's confidence in the predicting result 121 generated by the machine learning model 112.

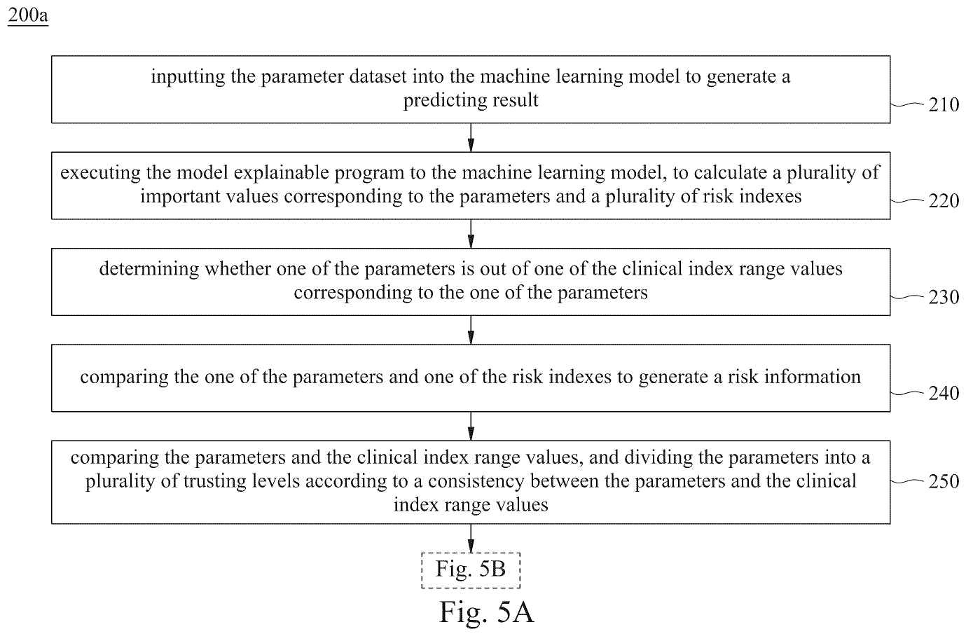

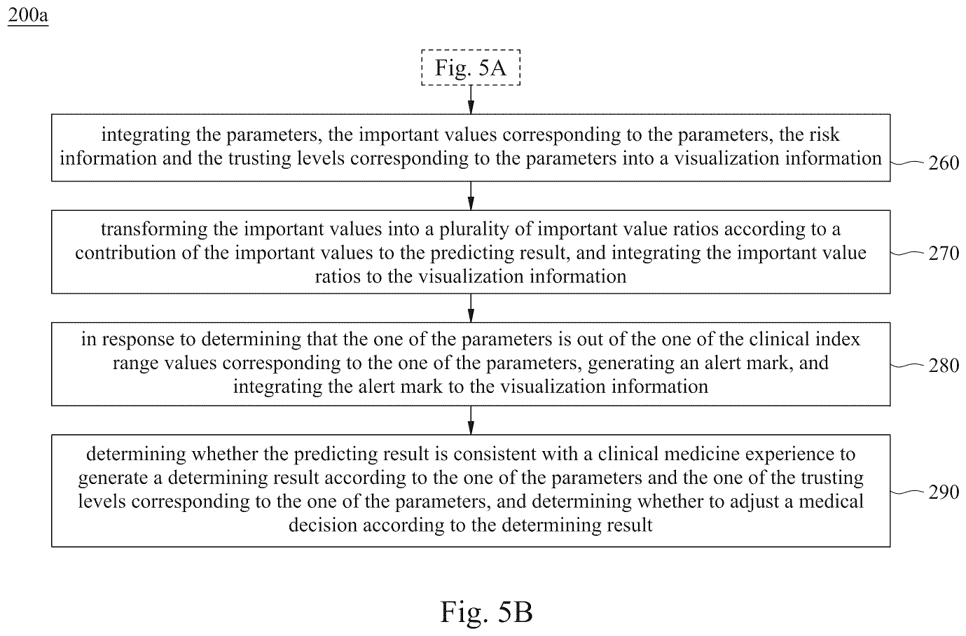

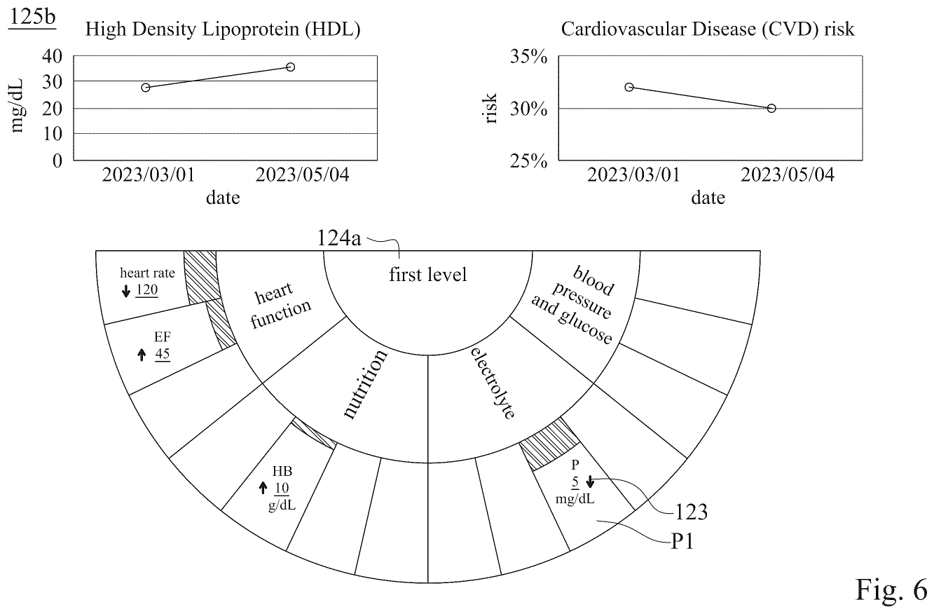

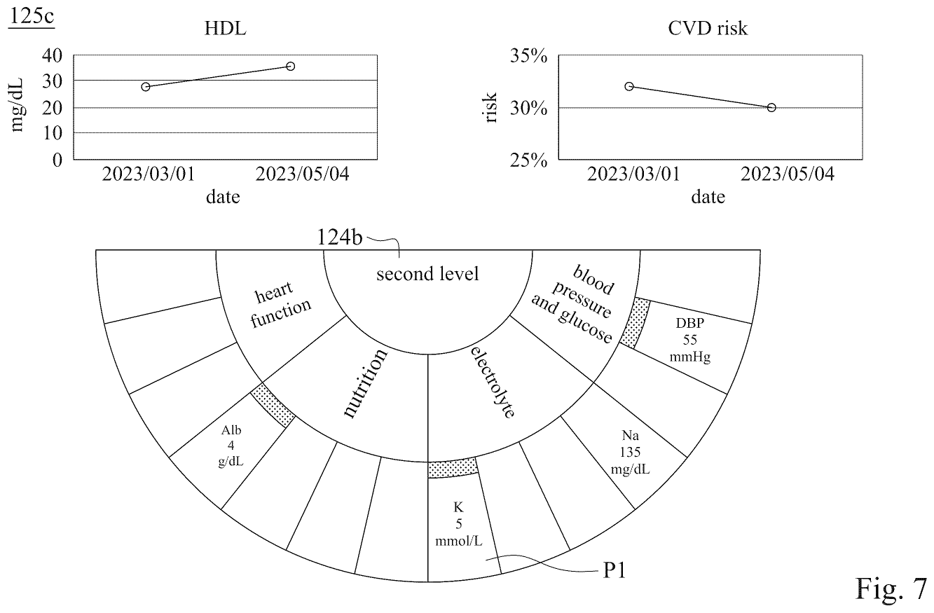

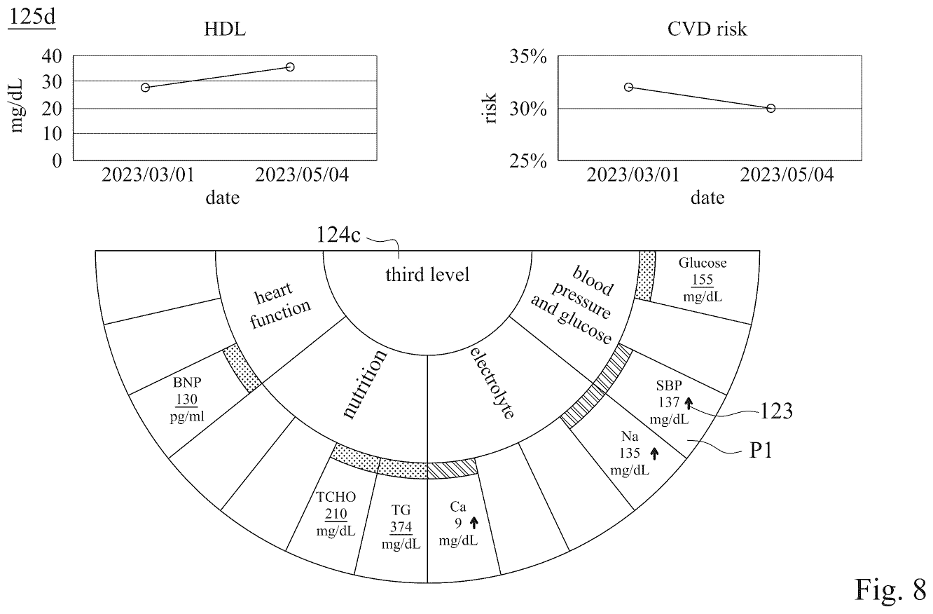

Please refer to FIG. 1, FIG. 5A to FIG. 8. FIG. 5A and FIG. 5B show flow charts of an explainable artificial intelligence method 200a applied to clinical medicine according to a third embodiment of the present disclosure. FIG. 6 shows a schematic view of a visualization information 125b generated by the explainable artificial intelligence method 200a applied to clinical medicine of FIG. 5A and FIG. 5B. FIG. 7 shows a schematic view of another visualization information 125c generated by the explainable artificial intelligence method 200a applied to clinical medicine of FIG. 5A and FIG. 5B. FIG. 8 shows a schematic view of further another visualization information 125d generated by the explainable artificial intelligence method 200a applied to clinical medicine of FIG. 5A and FIG. 5B. The explainable artificial intelligence method 200a applied to clinical medicine includes steps 210, 220, 230, 240, 250, 260, 270, 280, 290. In the third embodiment, the steps 210, 220, 230, 240, 250, 260 can be the same as the steps 210, 220, 230, 240, 250, 260 of the explainable artificial intelligence method 200 in the second embodiment, respectively, and will not be described again. The explainable artificial intelligence method 200a applied to clinical medicine can further include steps 270, 280, 290. The step 270 includes transforming the important values 122 into a plurality of important value ratios according to a contribution of the important values 122 to the predicting result 121, and integrating the important value ratios to the visualization information 125b, 125c, 125d. In the step 280, in response to determining that the one of the parameters P1 is out of the one of the clinical index range values 114 corresponding to the one of the parameters P1, generating an alert mark, and integrating the alert mark to the visualization information 125b, 125c, 125d. The step 290 includes determining whether the predicting result 121 is consistent with a clinical medicine experience to generate a determining result according to the one of the parameters P1 and the one of the trusting levels 124 corresponding to the one of the parameters P1, and determining whether to adjust a medical decision according to the determining result.

In detail, in the step 270, the important values 122 of all the parameters P1 are transformed into the important value ratios. Thus, the user can realize the importance ratio of all the parameters P1 directly while viewing the important value ratios of the parameters P1. In FIG. 6, the important value ratios can be shown in a hatched area above the column of one of the parameter P1, and different widths of the hatched areas represent the relative relationship of the important values 122 of the parameters P1.

In the step 280, the value of parameter P1, which is exceeded the clinical index range value 114, is underscored in FIG. 6 to FIG. 8. In other embodiments, the parameters can be marked in different colors to represent whether the parameter exceeding the clinical index range value or not.

In the step 290, if a parameter P1 is the first level 124a, the predicting result 121 can be viewed as high reliability, if a parameter P1 is the second level 124b, the predicting result 121 can be viewed as medium reliability, if a parameter P1 is the third level 124c, the predicting result 121 can be viewed as low reliability. Therefore, the explainable artificial intelligence method 200a applied to clinical medicine of the present disclosure can assist clinician to determine a reliability of the predicting result 121 accurately.

A non-transitory computer readable recording medium includes a program for the processor 120 capable of generating one of the visualization informations 125, 125a, 125b, 125c, 125d, to execute the explainable artificial intelligence method 200, 200a applied to clinical medicine. The non-transitory computer readable recording medium can be a CR-ROM, a flexible disk (FD), a CD-R, a digital versatile disk (DVD), a USB medium and a flash memory, but the present disclosure is not limited thereto.

According to the aforementioned embodiments and examples, the advantages of the present disclosure are described as follows.

1. The explainable artificial intelligence system applied to clinical medicine of the present disclosure can verify the predicting result generated by the machine learning model, thereby, avoiding a situation of wrong medical decision and treatment due to mistaken predicting result.

2. The explainable artificial intelligence method applied to clinical medicine of the present disclosure can enhance the physician's confidence in the predicting result generated by the machine learning model.

3. The explainable artificial intelligence method applied to clinical medicine of the present disclosure can assist clinician to determine a reliability of the predicting result accurately.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An explainable artificial intelligence method applied to clinical medicine, comprising:
driving a processor to read a parameter dataset, a machine learning model, a model explainable program and a plurality of clinical index range values from a database, wherein the parameter dataset comprises a plurality of parameters, and the clinical index range values are corresponding to the parameters, respectively;
driving the processor to input the parameter dataset into the machine learning model to generate a predicting result;
driving the processor to execute the model explainable program to the machine learning model, to calculate a plurality of important values correspond to the parameters and a plurality of risk indexes;
driving the processor to determine whether one of the parameters being out of one of the clinical index range values corresponding to the one of the parameters;
driving the processor to compare the one of the parameters and one of the risk indexes to generate a risk information;
driving the processor to compare the parameters and the clinical index range values, and divide the parameters into a plurality of trusting levels according to a consistency between the parameters and the clinical index range values, wherein the one of the parameters corresponds to one of the trusting levels; and
driving the processor to integrate the parameters, the important values corresponding to the parameters, the risk information and the trusting levels corresponding to the parameters into a visualization information.

2. The explainable artificial intelligence method applied to clinical medicine of claim 1, wherein the model explainable program is one of a SHapley Additive explanation (SHAP), a Local Interpretable Model-Agnostic Explanations (LIME) and an Individual Conditional Expectation (ICE).

3. The explainable artificial intelligence method applied to clinical medicine of claim 1, further comprising:
driving the processor to transform the important values into a plurality of important value ratios according to a contribution of the important values to the predicting result, and integrate the important value ratios to the visualization information.

4. The explainable artificial intelligence method applied to clinical medicine of claim 1, further comprising:
in response to determining that the one of the parameters being out of the one of the clinical index range values corresponding to the one of the parameters, driving the processor to generate an alert mark, and integrate the alert mark to the visualization information.

5. The explainable artificial intelligence method applied to clinical medicine of claim 1, further comprising:
driving the processor to determine whether the predicting result is consistent with a clinical medicine experience to generate a determining result according to the one of the parameters and the one of the trusting levels corresponding to the one of the parameters, and determine whether to adjust a medical decision according to the determining result.

6. An explainable artificial intelligence system applied to clinical medicine, comprising:
a database configured to access a parameter dataset, a machine learning model, a model explainable program and a plurality of clinical index range values, wherein the parameter dataset comprises a plurality of parameters, and the clinical index range values are corresponding to the parameters, respectively; and
a processor signally connected to the database, reading the parameter dataset, the machine learning model, the model explainable program and the clinical index range values and configured to perform an explainable artificial intelligence method applied to clinical medicine comprising:
inputting the parameter dataset into the machine learning model to generate a predicting result;
executing the model explainable program to the machine learning model, to calculate a plurality of important values correspond to the parameters and a plurality of risk indexes;
determining whether one of the parameters being out of one of the clinical index range values corresponding to the one of the parameters;
comparing a difference value between the one of the parameters and one of the risk indexes to generate a risk information;
comparing the parameters and the clinical index range values, and dividing the parameters into a plurality of trusting levels according to a consistency between the parameters and the clinical index range values, wherein the one of the parameters corresponds to one of the trusting levels; and
integrating the parameters, the important values corresponding to the parameters, the risk information and the trusting levels corresponding to the parameters into a visualization information.

7. The explainable artificial intelligence system applied to clinical medicine of claim 6, wherein the model explainable program is one of a SHapley Additive explanation, a Local Interpretable Model-Agnostic Explanations and an Individual Conditional Expectation.

8. The explainable artificial intelligence system applied to clinical medicine of claim 6, wherein the explainable artificial intelligence method applied to clinical medicine further comprises:
transforming the important values into a plurality of important value ratios according to a contribution of the important values to the predicting result, and integrate the important value ratios to the visualization information.

9. The explainable artificial intelligence system applied to clinical medicine of claim 6, wherein the explainable artificial intelligence method applied to clinical medicine further comprises:
in response to determining that the one of the parameters being out of the one of the clinical index range values corresponding to the one of the parameters, generating an alert mark, and integrating the alert mark to the visualization information.

10. The explainable artificial intelligence system applied to clinical medicine of claim 6, wherein the explainable artificial intelligence method applied to clinical medicine further comprises:
determining whether the predicting result is consistent with a clinical medicine experience to generate a determining result according to the one of the parameters and the one of the trusting levels corresponding to the one of the parameters, and determining whether to adjust a medical decision according to the determining result.

11. A non-transitory computer readable recording medium storing a program for a processor capable of generating a visualization information, to execute an explainable artificial intelligence method applied to clinical medicine comprising:
driving the processor to read a parameter dataset, a machine learning model, a model explainable program and a plurality of clinical index range values from a database, wherein the parameter dataset comprises a plurality of parameters, and the clinical index range values are corresponding to the parameters, respectively;
driving the processor to input the parameter dataset into the machine learning model to generate a predicting result;
driving the processor to execute the model explainable program to the machine learning model, to calculate a plurality of important values correspond to the parameters and a plurality of risk indexes;
driving the processor to determine whether one of the parameters being out of one of the clinical index range values corresponding to the one of the parameters;
driving the processor to compare the one of the parameters and one of the risk indexes to generate a risk information;
driving the processor to compare the parameters and the clinical index range values, and divide the parameters into a plurality of trusting levels according to a consistency between the parameters and the clinical index range values, wherein the one of the parameters corresponds to one of the trusting levels; and
driving the processor to integrate the parameters, the important values corresponding to the parameters, the risk information and the trusting levels corresponding to the parameters into the visualization information.

12. The non-transitory computer readable recording medium of claim 11, wherein the model explainable program is one of a SHapley Additive explanation, a Local Interpretable Model-Agnostic Explanations and an Individual Conditional Expectation.

13. The non-transitory computer readable recording medium of claim 11, wherein the explainable artificial intelligence method applied to clinical medicine further comprises:
driving the processor to transform the important values into a plurality of important value ratios according to a contribution of the important values to the predicting result, and integrate the important value ratios to the visualization information.

14. The non-transitory computer readable recording medium of claim 11, wherein the explainable artificial intelligence method applied to clinical medicine further comprises:
in response to determining that the one of the parameters being out of the one of the clinical index range values corresponding to the one of the parameters, driving the processor to generate an alert mark, and integrate the alert mark to the visualization information.

15. The non-transitory computer readable recording medium of claim 11, wherein the explainable artificial intelligence method applied to clinical medicine further comprises:
driving the processor to determine whether the predicting result is consistent with a clinical medicine experience to generate a determining result according to the one of the parameters and the one of the trusting levels corresponding to the one of the parameters, and determine whether to adjust a medical decision according to the determining result.
